(19) United States
(12) Patent Application Publication
(10) Pub. No.: US 2013/0012901 A1
(43) Pub. Date: Jan. 10, 2013

(54) LAMINATE POLYMER COMPOSITE WOUND DRESSINGS, THEIR MANUFACTURE AND THEIR USE

(75) Inventors: Nadi Ergenc, Tarrytown, NY (US); Peter Rudolf, Ladenburg (DE)

(73) Assignee: BASF Corporation, Florham Park, NJ (US)

(21) Appl. No.: 13/618,557

(22) Filed: Sep. 14, 2012

Related U.S. Application Data
(63) Continuation-in-part of application No. 13/406,034, filed on Feb. 27, 2012.
(60) Provisional application No. 61/505,700, filed on Jul. 8, 2011.
(60) Provisional application No. 61/448,076, filed on Mar. 1, 2011.

(51) Int. Cl. A61F 13/538

(52) U.S. Cl. 604/368

(57) ABSTRACT

A laminate polymer composite wound dressing and a process of making and using the same are provided. The laminate polymer composite wound dressing includes, a contact layer, an absorbent layer, and, optionally, a backsheet, an adhesive layer, and a moisture vapor transmission rate control layer, wherein the contact layer includes non-woven polymer fibers, the absorbent layer includes a fibrous web and super absorbent polymer particles. The laminate polymer composite wound dressing can find application in the medical field, such as the treatment of high exuding wounds and dry wounds.

### CROSS-REFERENCE TO RELATED APPLICATIONS

This application is a continuation-in-part application and claims the benefit of priority of U.S. application Ser. No. 13/406,034, filed on Feb. 27, 2012, which claims the benefit of priority of U.S. Provisional Application Ser. No. 61/448,076, filed on Mar. 1, 2011, and U.S. Provisional Application Ser. No. 61/505,700, filed on Jul. 8, 2011, all of which are herein incorporated by reference in their entireties.

### TECHNICAL FIELD

The present invention relates to a fluid absorbing polymer laminate composite, which can be used in the medical field, and in particular, as a wound dressing.

### BACKGROUND

In the medical field, there exists a need for improved wound dressings. The medical field has long suffered from problems related to chronic wounds, high exuding wounds, and bacterial infections, which necessitate the frequent changing of wound dressings. There is a need to avoid wetness, while keeping the wound bed moist to facilitate wound healing. There is also a need for wound fluid to be directed away from the wound while the fluid is locked into close proximity, but away from the wound bed, such that moisture is present at all times. Due to the nature of living skin, wound dressings are applied for a length of time sufficient to allow for skin to breath and moisture to pass through the wound dressing. Thus, wound dressings have been developed to absorb body fluids, while allowing air and moisture to pass through the material of the wound dressing. However, wound dressings have also been produced to completely cover the area of a wound in order to prevent abrasion and exclude bacteria. Thus, there is a need to completely cover a wound, while selectively allowing air and moisture to efficiently pass through the material of the wound dressing.

In order to provide sufficiently absorptive, breathable layers, wound dressings have been forced to incorporate a variety of trade-offs to accomplish these tasks. For example, wound dressings provide for absorbent layers that contain super absorbing materials. These super absorbing materials are capable of readily absorbing bodily fluids, allowing the wound to breathe, and protecting the wound from physical damage and/or bacteria. However, such super absorbent layers are often composed of materials that tend to adhere to the surface of the wound, lose their structural integrity upon becoming wet, and/or wick fluids from the wound site onto the surface of healthy skin. This permeation of a bodily fluid from the wound parallel to the surface of the wound dressing causes exposure of healthy skin to bodily fluids for as long as the wound dressing is in place. In turn, the exposure of healthy skin to bodily fluids over a long period of time induces maceration of healthy skin, causing the wound to grow.

Therefore, there exists a need for a wound dressing that is capable of preventing the wicking of bodily fluids from the wound site onto healthy skin.

Moreover, the biofouling of bodily fluids is a serious problem for wound dressings because the decomposition of bodily fluids in a wound dressing can lead to proliferation of infections and malodor. In practice, the biofouling of wound dressings, such as can occur with chronic wounds, necessitates that the wound dressings be changed frequently in order to prevent the proliferation of infections and malodor. However, if newly formed tissue (formed as a result of the healing process) adheres to the contact layer of the wound dressing, frequent changing of the wound dressing will impede healing because this newly formed tissue will be removed along with the contaminated wound dressing. Furthermore, the frequent changing of wound dressings requires higher labor costs. Thus, there is a need for the materials of the contact layer and the absorbent layer of a wound dressing to have antimicrobial properties that prevent or at least limit the ability of bacteria to grow and spread inside the wound dressing in order to reduce or eliminate the biofouling of the wound dressing and, thus, prevent the need for frequent changing of the wound dressing.

The present invention provides a solution to at least one of the above needs.

### SUMMARY

The following embodiments are not an extensive overview. The following drawings are not intended to either identify critical elements of the various embodiments, nor is it intended to the limit the scope of them. Additional variations will be apparent to the skilled person.

Embodiments of the present invention are directed to a laminate polymer composite wound dressing comprising a contact layer and an absorbent layer. The contact layer can comprise non-woven polymer fibers, and the absorbent layer can comprise a fibrous web and super absorbent polymer particles. The super absorbent polymer particles can be immobilized onto the fibrous web, and entanglement of at least some of the non-woven polymer fibers of the contact layer with at least some of the fibrous web of the absorbent layer binds a portion of the contact layer to a portion of the absorbent layer.

In one or more embodiments, there is a non-water soluble melt adhesive used to adhere at least a portion of the contact layer to at least a portion of the absorbent layer.

In a specific embodiment, there is no adhesive present between the contact layer and the absorbent layer. At least a portion of the non-woven fibers of the contact layer are entangled with at least a portion of the fibrous web of the absorbent layer.

In one or more embodiments, the non-woven polymer fibers of the contact layer comprise an ethylene-propylene copolymer, or a polyurethane of a polyether or a polyester. The non-woven polymer fibers have an elongation at break of 500% or more when measured at 73° F., when applied at a density of 1 to about 50 grams per square meter.

In one or more embodiments, the non-woven polymer fibers of the contact layer are applied at a density of about 2 to about 20 grams per square meter, and the ethylene-propylene copolymer has a glass transition temperature from about 110° F. to about 125° F.

In one or more embodiments, the fibrous web of the absorbent layer is comprised of a polyolefin, a polyester, a polyamide, a polyacrylate, or a mixture, copolymer, or blend thereof. The super absorbent particles of the absorbent layer are comprised of a cross-linked polymer formed from at least one monomer, wherein the at least one monomer is a carboxyl group-containing monomer, a carboxylic acid group-containing monomer, a carboxylic acid salt-containing monomer, a sulfonic acid group-containing polymer, a sulfonic acid salt group-containing monomer, hydroxyl group-containing monomer, an amide group-containing monomer, a quaternary ammonium salt group-containing monomer, or a copolymer thereof. The absorbent layer is capable of absorbing about 5 to about 80 times its dry weight of water or about 5 to about 20 times its dry weight in a saline solution.

In one or more embodiments, the absorbent layer is at least about two times more permeable to a fluid in a direction substantially perpendicular to the absorbent layer than in a direction substantially parallel to the absorbent layer.

In a specific embodiment, at least one of the contact layer and absorbent layer comprises as least one antimicrobial agent.

In a specific embodiment, the absorbent layer comprises an aqueous solution comprising at least one preservative and/or one or more polyols. At least one polyol is selected from glycerine, ethylene glycol, or propylene glycol. The preservative can be bronopol.

In one or more embodiments, the contact or absorbent layer is treated with at least one agent to support autolytic debridement.

In one or more embodiments the laminate polymer composite wound dressing comprises a contact layer, an absorbent layer, and a backsheet. At least a portion of the backsheet is located on an opposite side of the absorbent layer relative to the contact layer. The backsheet can comprise a moisture permeable material that has a moisture vapor transmission rate of from about 1.0 to about 3.0 kg/m²/day. The backsheet is permeable to air and substantially impermeable to a liquid and/or a bacteria.

In one or more embodiments, the laminate polymer composite wound dressing comprises a contact layer, an absorbent layer, a backsheet, and an adhesive layer. The adhesive layer comprises an adhesive. At least a portion of the adhesive layer is bound to at least a portion of the backsheet, and the adhesive layer is located between the absorbent layer and the backsheet. The adhesive layer is permeable to air, and optionally, contains at least one hole and/or is discontinuous.

In one or more embodiments, the laminate polymer composite wound dressing can further comprise a moisture vapor transmission rate control layer. The moisture vapor transmission rate control layer has a variable moisture vapor transmission rate and is located between the backsheet and the adhesive layer.

In one or more embodiments, the laminate polymer composite wound dressing can further comprise at least one removable layer that is in contact with the backsheet. The at least one removable layer comprises a moisture permeable material having a moisture vapor transmission rate of from about 1.0 to about 3.0 kg/m²/day and is permeable to air and substantially impermeable to a liquid and/or a bacteria.

In one or more embodiments, the laminate polymer composite wound dressing is sealed in a sterile package.

Other embodiments of the present invention are directed to a process for producing a laminate polymer composite wound dressing. The process comprises melt blowing a portion of a contact layer onto a portion of an absorbent layer. The contact layer comprises non-woven polymer fibers having a glass transition point. The absorbent layer comprised a fibrous web and super absorbent polymer particles. The melt blowing step is carried out at a temperature higher than the glass transition point of the non-woven polymer fibers of the contact layer.

In one or more embodiments, the process further comprises adhering a backsheet onto an adhesive layer, and adhering an adhesive layer onto the absorbent layer. Optionally, a moisture vapor transmission rate control layer can be adhered onto the adhesive layer.

In one or more embodiments, the process further comprises treating at least one of the contact layer and the absorbent layer with at least one of sterile water, a polyol, an antimicrobial agent, a preservative, or a mixture thereof.

In a specific embodiment, the process further comprises treating at least the absorbent layer with an aqueous solution comprising bronopol and/or one or more polyols. At least one polyol is selected from glycerine, ethylene glycol, or propylene glycol.

In one or more embodiments, the process comprises providing e-beam radiation in an energy range of from about 25 to about 35 kgrey.

In one or more embodiments, the process further comprises adhering at least one removable layer directly to the backsheet. At least one of the at least one removable layer comprises a moisture permeable material, wherein the moisture permeable material has a moisture vapor transmission rate of from about 1.0 to about 3.0 kg/m²/day and is permeable to air and substantially impermeable to a liquid and/or a bacteria.

In another aspect, embodiments of the invention pertain to a laminate polymer composite wound dressing for placement over a wound comprising: a contact layer comprising non-woven polymer fibers; an absorbent layer comprising a fibrous web and super absorbent polymer particles that form a gel upon contact with liquid wound exudate, the absorbent layer being expandable and more permeable to a fluid in a direction substantially perpendicular to the wound than in a direction substantially parallel/horizontal to wound, the absorbent layer being able to expand in a direction perpendicularly to the wound to enable the wound dressing to conform to a wound having a varying depth; a backsheet, wherein at least a portion of the backsheet is located on an opposite side of the absorbent layer relative to the contact layer, and wherein the backsheet comprises a moisture permeable material, wherein the backsheet is permeable to air and substantially impermeable to a liquid and/or a bacteria; and a moisture vapor transmission rate control layer, disposed between the absorbent layer and the backsheet, wherein when the moisture vapor transmission rate control layer is wet with liquid wound exudate, moisture permeates through the moisture vapor transmission rate control layer, and when the moisture vapor transmission rate control layer is dry, no vapor transmits through the moisture vapor transmission rate control layer. In one embodiment, the fibrous web comprises a polyester, and the super absorbent polymer particles comprise polyacrylate. The laminate polymer composite wound dressing can further include at least one removable layer, wherein removal of at least a portion of the removable layer results in a moisture vapor transmission rate for the polymer composite that increases or decreases the moisture vapor transmission rate prior to removal of the removable layer. The at least one removable layer can have a moisture vapor transmission rate of from 1.0 to 3.0 kg/m²/day. In an embodiment, at least portion of the removable layer comprises a strip. In an embodiment, the backsheet material has a moisture vapor transmission rate of from 1.0 to 3.0 kg/m²/day. In an embodiment, the moisture vapor control layer is selected from polyvinyl alcohol, polyethylene oxide, polyvinyl pyrrolidone and blends or copolymers thereof. In an embodiment, the absorbent layer is at least about two times more permeable to a fluid in a direction substantially perpendicular/vertical to the absorbent layer than in a direction substantially parallel/horizontal to the absorbent layer. In an embodiment, the moisture vapor transmission rate control layer has a moisture vapor transmission rate exceeding 1000 g/m²/24 hours when the wound dressing is in contact with a highly exuding wound. In an embodiment, the moisture vapor transmission rate control layer has a moisture vapor transmission rate that varies depending upon the type of a wound being treated. In an embodiment, removal of the removable layer results in reduced a moisture vapor transmission rate of the composite so that a low exuding wound can be kept moist. In an embodiment, the absorbent layer contains water and glycerin. In an embodiment, the absorbent layer comprises an antimicrobial. In an embodiment, the backsheet comprises polyurethane.

### BRIEF DESCRIPTION OF THE DRAWINGS

For the purpose of illustrating the embodiments disclosed therein, there are depicted in the drawings certain embodiments of a laminate polymer composite wound dressing. However, the methods and related products are not limited to the precise arrangements and instrumentalities of the embodiments depicted in the drawings.

FIG. 1 schematically depicts a cross-section view of the absorbent layer of the wound dressing.

FIG. 2 schematically depicts a cross-section view of an embodiment of a laminate polymer composite wound dressing. The laminate polymer composite wound dressing includes a contact layer and an absorbent layer.

FIG. 3 schematically depicts a cross-section view of an embodiment of a laminate polymer composite wound dressing. The laminate polymer composite wound dressing comprises an absorbent layer located between a contact layer and a backsheet.

FIG. 4 schematically depicts a cross-section view of an embodiment of a laminate polymer composite wound dressing. The laminate polymer composite wound dressing comprises a contact layer, an absorbent layer, an adhesive layer, and a backsheet.

FIG. 5 schematically depicts a cross-section view of an embodiment of a laminate polymer composite wound dressing. The laminate polymer composite wound dressing comprises a contact layer, an absorbent layer, an adhesive layer, a moisture vapor transmission control layer, and a backsheet

FIG. 6 schematically depicts a cross-section view of an embodiment of a laminate polymer composite wound dressing. The laminate polymer composite wound dressing comprises a contact layer, an absorbent layer, an adhesive layer, a moisture vapor transmission layer, and at least one removable layer adjacent to the backsheet.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

As envisioned in the present invention with respect to the disclosed laminate polymer composite wound dressings and methods, in one aspect the embodiments comprise the components and/or steps disclosed therein. In another aspect, the embodiments consist essentially of the components and/or steps disclosed therein. In yet another aspect, the embodiments consist of the components and/or steps disclosed therein.

With respect to terms used in this disclosure, the following definitions are provided.

The articles “a” and “an” are used herein to refer to one or more than one object of the article. By way of an example, “an element” means one or more than one element.

The term “about” will be understood by persons of ordinary skill in the art to depend on the context in which it is used. As used herein, “about” is meant to encompass variations from ±20%, including ±10%, ±5%, ±1%, and ±0.1%.

It is understood that any and all, whole or partial integers between any ranges set forth herein are included.

As used herein, in one or more embodiments, the term “bind” or “bound” refers to the physical entanglement of one object to another. For example, two adjacent surfaces of a clean material may be made to bind to the other through many means known in the art, including physical entanglement of adjacent materials.

As used herein, in one or more embodiments, the term “laminate polymer composite” means at least one polymer layer bound to at least one other polymer layer, such that the layers of the polymer laminate composite extend in two dimensions (e.g., length and width) and has a thickness that is smaller than the length and/or width of the layer.

As used herein, in one or more embodiments, the term “substantially” means over 90%, including over 95%, including over 98%.

As used herein, in one or more embodiments, the term “portion” means less than a whole, including more than 50% of the whole, including more than 75% of the whole.

As used herein, in one or more embodiments, the term “parallel” in the context of the laminate polymer composite means along the plane generally defined by the length and width of the laminate polymer composite, and wherein the “parallel” direction is orthogonal to the thickness of the film.

As used herein, in one or more embodiments, the term “perpendicular” in the context of the laminate polymer composite wound dressing means in the direction of the thickness of the wound dressing and wherein the term “perpendicular” is orthogonal to the plane generally defined by the length and width of a layer of the laminate polymer composite wound dressing.

As used herein, in one or more embodiments, the term “super absorbing” or “super absorbent” refers to a property of a material, wherein the material can absorb a large amount of a liquid relative to the weight of the material. For example, a super absorbing material can absorb from about 0 to about 80 times its weight in water its weight in water. As an additional example, a super absorbing material can absorb from about 0-20 times its weight in a saline solution, wherein the saline solution is a 0.8% solution of water and sodium chloride. It should be noted that the term “super absorbent” and “super absorbing” can be used interchangeably, unless otherwise noted.

As used herein, in one or more embodiments, the term “non-woven” refers to a material made of at least one fiber, wherein the at least one fiber is bonded together by a chemical treatment, mechanical treatment, heat treatment, solvent treatment, entanglement, and the like, or combination thereof.

As used herein, in one or more embodiments, the term “permeable” refers to the ability of a permeate, such as a liquid, gas, or vapor, to pass through a material. For example, a layer of material is permeable to liquid to the extent that the layer of material allows the liquid to pass through the material.

As used herein, in one or more embodiments, the term “permeability” means the rate at which a specific permeate, such a given liquid, gas, vapor or class thereof, is capable of passing through a material. For example, the permeability of a material may be measured as the rate at which a permeate, such as a liquid, passes through a surface area, under a pressure difference, considering the thickness of the material. For example, permeability can be measured in units of cm³·mm/(m²·Bar·24 hours).

As used herein, in one or more embodiments, the term “bodily fluid” means any liquid originating from inside the body of a living person or animal, including such fluids as blood, all the contents of blood, and exudates from wounds. When a wound is moist or wet, moisture tends to escape in a vapor form (i.e. water vapor). Thus, “bodily fluid” also includes water vapor.

As used herein, in one or more embodiments, the term “absorb” refers to the process of a liquid being trapped in a material. The term “absorbent” means a material that is capable of absorbing a liquid.

As used herein, in one or more embodiments, the term “entanglement” refers to a physical arrangement, wherein at least one part of a material extends into or around another layer of material such that the two materials become physically bound together without the use of an adhesive. For example, entanglement can occur where a material, in the form of a fiber, is physically penetrated into or tied around the material of another layer, such that the material of the fiber is bound to the layer.

As used herein, in one or more embodiments, the terms “entangle” and “entangled” refer to the act of physically arranging materials such that at least one part of a material extends into or around another layer of material so as to physically bind the two layers together.

As used herein, in one or more embodiments, a “fiber” is a form of a material wherein the diameter or thickness is less than about 500 micrometers.

As used herein, in one or more embodiments, a “microfiber” is a form of a material wherein the diameter or thickness is less than about 50 micrometers. A microfiber is, by definition, a fiber, but a fiber is not necessarily a microfiber.

As used herein, in one or more embodiments, the term “fibrous web” refers to a layer of fibers that can be woven or unwoven.

As used herein, in one or more embodiments, the term “immobilized” refers to state of being where one object is chemically bonded or physically bound to another object, such that the first object cannot be moved without also moving the second object.

As used herein, in one or more embodiments, the term “copolymer” refers to the formation of a polymer using two or more different non-identical monomers.

As used herein, in one or more embodiments, the term “discontinuous” refers to a material that is arranged so that at least a part of the material is not connected to or in contact with another part of the material. For example, if a material were arranged into a sheet and one part of the sheet was physically separated from the rest of the sheet, then the sheet could be described as discontinuous.

As used herein, in one or more embodiments, the term “hole” means a space or gap in a material such that none of the material may be found therein.

As used herein, in one or more embodiments, the term “moisture vapor transmission rate” refers to the measure of the passage of water vapor through a substance. Moisture vapor transmission rate can be measured by a variety of gravimetric techniques and can be expressed in units of grams/meters squared/24 hours (g/m²/24 hours) or grams/100 inches squared/24 hours or kilograms/meters squared/day (kg/m²/day).

As used herein, in one or more embodiments, the term “melt blowing” means the process of extruding a polymer through a series of die nozzles into a high velocity hot air stream to form fibers.

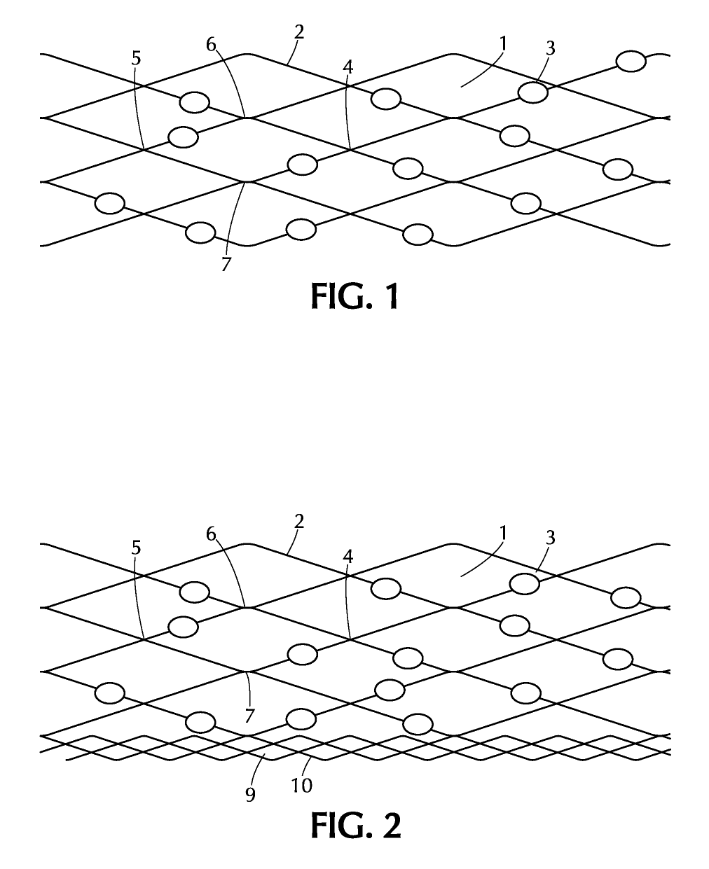

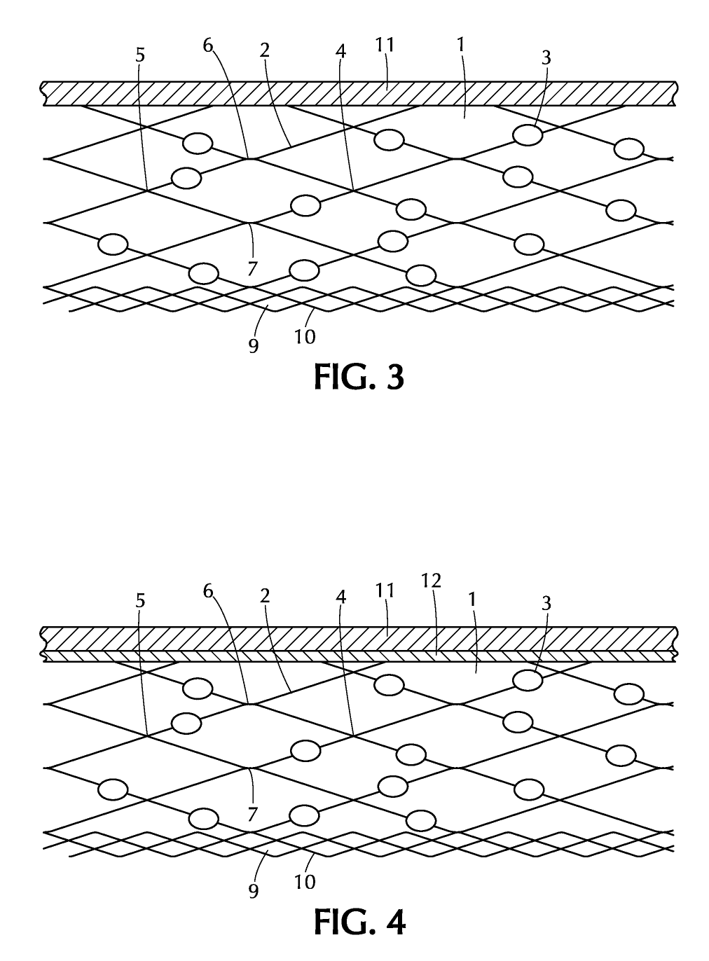

Provided are laminate polymer composite wound dressings comprising a contact layer comprising non-woven polymer fibers, and an absorbent layer comprising a fibrous web and super absorbent polymer particles. Entanglement of the non-woven polymer fibers of the contact layer and the fibrous web of the absorbent layer can function to bind a portion of the contact layer to a portion of the absorbent layer. A non-water soluble melt adhesive can also be used to adhere a portion of the contact layer to a portion of the absorbent layer. The non-water soluble melt adhesive can comprise a polyurethane adhesive. The absorbent layer is shown in FIG. 1, and the assembled laminate polymer composite wound dressing is shown in FIG. 2. FIGS. 3 to 6 illustrate alternative embodiments of the present invention.

FIG. 1 provides a schematic representation of a cross-section view of an absorbent layer 1 of a laminate polymer composite wound dressing. The absorbent layer 1 comprises a fibrous web 2 and super absorbent polymer particles 3. The super absorbent polymer particles 3 are distributed throughout the absorbent layer 1 and are integral with the fibrous web 2. The fibrous web 2 is continuous, mostly oriented along the absorbent layer 1 and intersected with each other. The distance between horizontally adjacent intersection points 4 and 5 of the fibrous web 2 is larger than the distance between the vertically adjacent intersection points 6 and 7. The difference in the horizontal distance versus the vertical distance permits vertical expansion of the absorbent layer 1 upon exposure to wound exudates.

FIG. 2 provides a schematic representation of a cross-section view of a laminate polymer composite wound dressing comprising an absorbent layer 1 and a contact layer 9. The absorbent layer 1 comprises a fibrous web 2 and super absorbent polymer particles 3. The super absorbent polymer particles 3 are distributed throughout the absorbent layer 1 and are integral with the fibrous web 2. The fibrous web 2 is continuous, mostly oriented along the absorbent layer 1 and intersected with each other. The distance between horizontally adjacent intersection points 4 and 5 of the fibrous web 2 is larger than the distance between the vertically adjacent intersection points 6 and 7. The difference in the horizontal distance versus the vertical distance permits vertical expansion of the absorbent layer 1 upon exposure to wound exudates. The contact layer 9 comprises non-woven polymer fibers 10. Entanglement of the non-woven polymer fibers 10 of the contact layer 9 and the fibrous web 2 of the absorbent layer 1 binds a portion of the contact layer 9 to a portion of the absorbent layer 1 without the use of adhesives. For example, the non-woven polymer fibers 10 can be melt blown to the absorbent layer 1 without the use of adhesives. Optionally, a non-water soluble melt adhesive can be used to adhere the contact layer 9 to the absorbent layer 1.

In an embodiment, the contact layer can comprise non-woven polymer fibers. A function of the contact layer can be to prevent or reduce adhesion between the absorbent layer to a substrate, wherein the substrate can be a biological tissue, such as a wound in dermal tissue. To ensure low adhesion to tissue, the fibers can be non-polar or only partially hydrophilic. An additional function of the contact layer can be to direct fluid into the absorbent layer and away from healthy skin. Polymers suitable for making the non-woven fibers of the first layer can have elastic properties and a softening point below 125° F.

The material for the non-woven polymer fibers is not particularly limited so long as the material can be bound to the absorbent layer without the use of adhesives. For example, polymers suitable for the non-woven fibers can be melt blown to bind to another layer without the use of adhesives. A suitable material for non-woven polymer fibers can include a polyolefin, an ethylene-propylene copolymer, or a polyurethane of a polyether or a polyester. Further, the polyurethane of a polyether or a polyester can be characterized by a glass transition temperature from about −60° F. to about 0° F. The ethylene-propylene copolymer can have a glass transition temperature from about 110° F. to about 125° F. The non-woven polymer fibers can be applied at a density of about 1 to about 50 grams per square meter, including about 2 to about 20 or 2 to about 15 grams per square meter. The non-woven polymer fibers can have an elongation at break of 500% or more when measured at 73° F. The non-woven polymer fibers can be a polyurethane of a polyester or a polyether, such as Elastollan® B95A11N, Elastollan® P9291 or Elastollan® 1100 series (BASF). The non-woven polymer fibers can be an ethylene-propylene copolymer, including but not limited to propylene based elastomers such as Vistamaxx®3000. Vistamaxx®3020FL, Vistamaxx®2330, Vistamaxx®3980FL, Vistamaxx®6102, Vistamaxx®6102FL, Vistamaxx®6202, Vistamaxx®6206FL, Vistamaxx®2320, and Vistamaxx®2330.

A benefit of the choice of the method and the material for the first layer (i.e., the contact layer) can be the reduction or prevention of delamination of the wound dressing, especially upon absorption of bodily fluids. Adhesives are often used to adhere the contact layer to the absorptive layer. However, if a water-soluble adhesive is used, upon contact with bodily fluids, the water-soluble adhesive can dissolve and disintegrate, causing the wound dressing to delaminate. It is a major challenge, however, to attach or bind layers onto a super absorbing polymer layer because the super absorbing polymer layer generally expands upon contact with a fluid. This expansion leads to insufficient physical integrity. A solution to this problem can be the direct application of a melt-blown layer, such as the contact layer, onto a super absorbing polymer containing material (fabric), such as the absorbent layer. A benefit to this choice of material is that the wound dressing does not delaminate upon absorption of bodily fluids. Alternatively, a non-water soluble melt adhesive, such as, but not limited to, a polyurethane adhesive, can be used to adhere the contact layer to at least a portion of the absorbent layer. The non-water soluble melt adhesive will not dissolve and disintegrate upon exposure to bodily fluid, thus preventing the problem of delamination of the wound care dressing.

The step of adhering the contact layer to the absorbent layer can depend on the type of the adhesive used. For example, heat, pressure, and/or light may be applied to the layers or to the adhesive to facilitate adhesion between the layers.

In an embodiment, the contact layer includes a material which is characterized by high elasticity and low glass transition points in order to provide sufficient physical integrity without the need for an adhesive. The elasticity characteristic of the material of the contact layer provides a mechanism for the material to bind to the absorbent layer. Upon absorption of bodily fluids, which leads to the expansion of the absorbent layer, the elasticity of the contact layer prevents delamination. Optionally, a non-water soluble melt adhesive can be used to adhere the contact layer to the absorbent layer.

A further benefit of the choice of the method and the material for the contact layer is that it prevents loose super absorbing polymer (SAP) particles within an absorbent layer from detaching and adhering to the wound. One method for limiting detachment of super absorbing polymer particles is to place the absorbent layer between two layers that prevent migration of small particles.

In a further embodiment, the absorbent layer comprises a fibrous web and super absorbent polymer particles, wherein the super absorbent polymer particles can be immobilized onto the fibrous web. It is important that a wound dressing is flexible so that the wound dressing can conform in shape/depth and/or adjust to any shape or contour of a wound bed. By nature, however, super absorbing particles are brittle and inflexible. Having discrete super absorbing polymer particles immobilized on a flexible fibrous web will render the entire laminate polymer composite wound dressing flexible. The fibrous web can be made of synthetic fibers. Examples of synthetic fibers for the fibrous web include fibers comprising at least one of a polyolefin, such as polyethylene, polypropylene, and the like; a polyester, such as polyethylene terephthalate and the like; a polyamide, such as nylon 6, nylon 6,6, poly(amino carboxyl pentamethylene) and the like; a polyacrylate, such as poly(acrylic acid), poly(methacrylic acid), poly(acrylic acid sodium salt) and poly(methacrylic acid sodium salt). The synthetic polymeric fibers can be formed by melt blowing, through a spunbond process, by extrusion and drawing, or other wet, dry and melt spinning methods known to those skilled in the art. The fibrous web including synthetic fibers has a basis weight of from about 20 to about 200, including of from about 30 to about 150, including of from about 35 to about 125 grams per square meter. The fibrous web including synthetic fibers suitably has a density of from about 0.005 to about 0.12, including of from about 0.008 to about 0.1, including of from about 0.01 to about 0.08 gram per cubic centimeter.

The absorbent layer can be manufactured in a continuous process by spraying liquid monomer solutions onto an inert fabric, as a first step; by cure or crosslinking of the monomers, in a second step; and drying of the final super absorbent fabric, in a third step. Curing or crosslinking the monomers on the inert fabric can produce immobilized, discrete super absorbent droplets on the fabric.

In a further embodiment, monomers can be sprayed on both sides of the inert fabric to produce an absorbent layer that shows improved properties with respect to a bodily fluid being able to travel perpendicularly/vertically as opposed to laterally or parallel or horizontal.

The super absorbent polymer particles are comprised of a cross-linked polymer formed from at least one monomer, wherein the at least one monomer is a carboxyl group containing monomer, a carboxylic acid group-containing monomer, a carboxylic acid salt containing monomer, a sulfonic acid group-containing polymer, a sulfonic acid salt group containing monomer, hydroxyl group-containing monomer, an amide group-containing monomer, a quaternary ammonium salt group-containing monomer, or a copolymer thereof. Examples of suitable superabsorbent forming monomers are as follows: (i) Carboxyl group containing monomers: monoethylenically unsaturated mono or poly-carboxylic acids, such as (meth)acrylic acid (meaning acrylic acid or methacrylic acid. Similar notations are used hereinafter), maleic acid, fumaric acid, crotonic acid, sorbic acid, itaconic acid, and cinnamic acid; (ii) Carboxylic acid anhydride group-containing monomers: monoethylenically unsaturated polycarboxylic acid anhydrides (such as maleic anhydride); (iii) Carboxylic acid salt-containing monomers: water-soluble salts (alkali metal salts, ammonium salts, amine salts, etc.) of monoethylenically unsaturated mono- or poly-carboxylic acids (such as sodium(meth)acrylate, trimethylamine(meth)acrylate, triethanolamine(meth)acrylate, sodium maleate, methylamine maleate); (iv) Sulfonic acid group-containing monomers: aliphatic or aromatic vinyl sulfonic acids (such as vinylsulfonic acid, allyl sulfonic acid, vinyltoluenesulfonic acid, styrene sulfonic acid), (meth)acrylic sulfonic acids [such as sulfopropyl(meth)acrylate, 2-hydroxy-3-(meth)acryloxy propyl sulfonic acid]; (v) Sulfonic acid salt group-containing monomers: alkali metal salts, ammonium salts, amine salts of sulfonic acid group-containing monomers as mentioned above; (vi) Hydroxyl group containing monomers: monoethylenically unsaturated alcohols (such as (meth)allyl alcohol), monoethylenically unsaturated ethers or esters of polyols (alkylene glycols, glycerol, polyoxyalkylene polyols), such as hydroxethyl(meth)acrylate, hydroxypropyl(meth)acrylate, triethylene glycol(meth)acrylate, poly(oxyethylene oxypropylene)glycol mono(meth)allyl ether (in which hydroxyl groups may be etherified or esterified); (vii) Amide group-containing monomers: vinylformamide, (meth)acrylamide, N-alkyl(meth)acrylamides (such as Nmethylacrylamide, N-hexylacrylamide), N,N-dialkyl(meth)acryl amides (such as N,Ndimethylacrylamide, N,N′-di-n-propylacrylamide), N-hydroxyalkyl(meth)acrylamides (such as N-methylol(meth)acrylamide, N-hydroxyethyl(meth)acrylamide), N,N-dihydroxyalkyl (meth)acrylamides (such as N,N-dihydroxyethyl(meth)acrylamide), vinyllactams (such as Nvinylpyrrolidone); (viii) Amino group-containing monomers: amino group-containing esters (e.g. dialkylaminoalkyl esters, dihydroxyalkyl aminoalkyl esters, morpholinoalkyl esters, etc.) of monoethylenically unsaturated mono- or di-carboxylic acid (such as dimethlaminoethyl(meth)acrylate, diethylaminoethyl(meth)acrylate, morpholinoethyl(meth)acrylate, dimethyl aminoethyl fumarate), heterocyclic vinyl compounds (such as vinyl pyridines {e.g. 2-vinyl pyridine, 4-vinyl pyridine, N-vinyl pyridine} and N-vinyl imidazol); and (ix) Quaternary ammonium salt group-containing monomers: N,N,N-trialkyl-N(meth)acryloyloxyalkylammonium salts (such as N,N,N-trimethyl-N-(meth)acryloyl oxyethylammonium chloride, N,N,N-triethyl-N-(meth)acryloyl oxyethylammonium chloride, 2-hydroxy-3-(meth)acryloyloxypropyl trimethyl ammonium chloride).

In another embodiment, the super absorbent polymer particles can be comprised of a cross-linked polymer formed from at least one monomer, wherein the at least one monomer can be as follows: acrylic acid, methacrylic acid, maleic acid, fumaric acid, crotonic acid, sorbic acid, itaconic acid, cinnamic acid, vinyl sulfonic acid, allyl sulfonic acid, vinyl toluene sulfonic acid, styrene sulfonic acid, sulfo(meth)acrylate, sulfopropyl(meth)acrylate, 2-acrylamide-2-methylpropane sulfonic acid, 2-hydroxyethyl(meth)acryloylphosphate, phenyl-2-acryloyloxyethylphosphate, or the sodium, potassium and ammonium salts thereof, or maleic anhydride, and combinations thereof.

In a further embodiment, the absorbent layer can absorb about 0 to about 80 times, including about 5 to about 80 times, including about 20 to about 60 times, its dry weight of water. The absorbent layer can absorb about 0 to about 20 times, including about 5 to about 20 times, including about 10 to about 15 times, its dry weight in a saline solution, wherein the saline solution is a 0.8% solution of water and sodium chloride. More particularly, the absorbent layer can absorb from about 0 g/cm² to about 0.9 g/cm² of a saline solution, wherein the saline solution is a 0.8% solution of water and sodium chloride. The method used to measure the absorption of the absorbent layer is to compare the weight of the absorbent layer when dry against the weight of the absorbent layer after water or saline solution has been added to the absorbent layer. In an embodiment, the absorbent layer is at least about two times more permeable to a fluid in a direction substantially perpendicular/vertical to the absorbent layer (i.e., the wound) than in a direction substantially parallel/horizontal to the absorbent layer (i.e., the wound). This feature allows the wound dressing to conform to the varying shape/depth of the wound bed. For example, a wound is often irregular in shape and/or depth, with some portions of the wound being deeper and/or shallower. Because the absorbent layer is able to expand perpendicularly/vertically, the wound dressing is able to conform and expand in the vertical direction. This prevents pools of wound exudates from forming, which facilitates faster wound healing. At the same time, lateral or horizontal expansion along the wound surface is minimized, which prevents the wound care dressing from macerating healthy tissue.

In one embodiment, the absorbent layer can be comprised of Luquafleece® (BASF, Florham Park, N.J.).

Exemplary materials of the absorbent layer and the methods used to make the second layer are discussed in detail in U.S. Pat. No. 6,417,425, which is incorporated herein by reference.

In another embodiment, the contact layer comprises at least one antimicrobial agent. The antimicrobial agent will prevent microorganisms from growing on the contact layer of the wound dressing. In another embodiment, the absorbent layer comprises at least one preservative incorporated into the super absorbing polymer layer. The preservative serves to prevent the growth of microorganisms, including fungi, in the absorbent layer. A benefit of the laminate polymer composite wound dressing can be the inclusion of such one or more active antimicrobial or preservative agents in the contact and/or absorbent layers to stop, prevent, or slow biofouling of the laminate polymer composite wound dressing.

The wound dressing, via the contact layer and/or the absorbent layer, may comprise up to about 10% by weight, for example from about 0.01 to about 5% by weight, typically from about 0.1 to about 2% by weight of one or more antimicrobials. Examples of antimicrobial agents include but are not limited to triclosan (5-chloro-2-(2,4-dichlorophenoxy)phenol), poly(hexamethylene biguanide) (PHMB), silver, silver salts, or mixtures thereof. The antimicrobial agent is not particularly limited so long as it has the property of killing, preventing, or limiting the growth of bacteria and other infectious diseases in the first layer and/or a second layer.

In an embodiment, it can be advantageous for the absorbent layer, and, therefore, the entire laminate polymer composite wound dressing, to be moisturized prior to placing the wound dressing onto a wound. Pre-moisturizing with any aqueous fluid, such as saline or sterile water, can render the polymer laminate more flexible, and, thus, more adaptable to the shape/depth of the wound bed. In an embodiment, at least the absorbent layer comprising a fibrous web and super absorbent polymer particles is treated with a solution of water or saline, a preservative, such as, but not limited to, bronopol (2-bromo-2-nitropropane-1,3-diol), and/or at least one polyol. In an embodiment, the absorbent layer is treated with about 0 to about 0.5 g/cm² of water, and more preferably about 0.05 g/cm² of water. In a further embodiment, the absorbent layer is treated with about 0 to about 0.9 g/cm² of saline solution, and more preferably about 0.78 g/cm² of saline solution. Treatment with water or saline solution moistens the wound dressing without significantly impacting the absorption capacity of the absorbent layer. Additionally, the moisture provided by the water or saline solution allows the wound dressing to be used on dry wounds to support autolytic debridement, in addition to use on high exuding wounds. In an embodiment, the absorbent layer is treated with about 0 to about 0.1 g/cm² of at least one polyol. Exemplary polyols include, but are not limited to, glycerine, ethylene glycol, propylene glycol, and combinations thereof. Treatment with a polyol supports softness of the wound dressing even when dry. In an embodiment, the absorbent layer is treated with about 0 to about 0.1% of a preservative, such as bronopol. The bronopol acts as a preservative. Other suitable preservatives can be used either alone or with at least one polyol. Treatment with a preservative preserves the absorbent layer by preventing or limiting growth of microorganisms, including fungi, which aides in suppression of malodor and proliferation of infection. Thus, in an embodiment, at least the absorbent layer of the laminate polymer composite wound dressing can comprise an aqueous solution of bronopol and/or one or more polyols, wherein at least one polyol is selected from glycerine, ethylene glycol or propylene glycol. In some embodiments, the polyol is selected from the group consisting of glycerine, ethylene glycol, propylene glycol, and combinations thereof. The water or saline solution and one or more polyols can render the super absorbing second layer smooth, soft, and flexible. Adding fluid to the composite material or to the absorbent layer in particular can also be advantageous for dry, non-exuding wounds. For dry, non-exuding wounds, a function of the absorbent layer can be to serve as a source of moisture, which can be essential for wound healing.

While fluid absorption capacity of the laminate polymer composite wound dressing can be important, it can also be important that the bodily fluids are trapped in the absorbent layer, so that leakage can be prevented and lateral spreading of bodily fluids can be minimized Avoiding or minimizing maceration, therefore, can be a combination of the design and choice of materials of the contact layer and absorbent layer. Ensuring physical integrity of the entire laminate polymer composite wound dressing can be important in any use of the laminate polymer composite wound dressing for treating highly exuding wounds.

In an embodiment, the laminate polymer composite wound dressing can be sealed in a sterile package.

FIG. 3 provides a schematic representation of a cross-section view of a laminate polymer composite wound dressing comprising an absorbent layer 1 interposed between a contact layer 9 and a backsheet 11. The absorbent layer 1 comprises a fibrous web 2 and super absorbent polymer particles 3. The super absorbent polymer particles 3 are distributed throughout the absorbent layer 1 and are integral with the fibrous web 2. The fibrous web 2 is continuous, mostly oriented along the absorbent layer 1 and intersected with each other. The distance between horizontally adjacent intersection points 4 and 5 of the fibrous web 2 is larger than the distance between the vertically adjacent intersection points 6 and 7. The difference in the horizontal distance versus the vertical distance permits vertical expansion of the absorbent layer 1 upon exposure to wound exudates. The contact layer 9 comprises non-woven polymer fibers 10. Entanglement of the non-woven polymer fibers 10 of the contact layer 9 and the fibrous web 2 of the absorbent layer 1 binds a portion of the contact layer 9 to a portion of the absorbent layer 1 without the use of adhesives. For example, the non-woven polymer fibers 10 can be melt blown to the absorbent layer 1 without the use of adhesives. Optionally, a non-water soluble melt adhesive can be used to adhere the contact layer 9 to the absorbent layer 1. At least a portion of the backsheet 11 can be located on an opposite side of the absorbent layer 1 relative to the contact layer 9. The backsheet 11 comprises a moisture permeable material.

The moisture permeable material can have a moisture vapor transmission rate of from about 1.0 to about 3.0 kg/m²/day. The moisture permeable material can also be permeable to air and can be substantially impermeable to a liquid and/or a bacteria. The backsheet can be a moisture permeable polyurethane material having a maximum pore size, which is smaller than the size of a microorganism, such as bacteria. The backsheet can be oxidized by a corona treatment or the like to facilitate adhesion to another layer, such as an adhesive layer.

An advantage of using a moisture permeable material having high moisture vapor transmission rate (MVTR) of >1 kg water/m²/day can be that the backsheet serves as an effective barrier, which prevents microorganisms from penetrating into the dressing. Further, the moisture permeable material can allow moisture to evaporate. The evaporation of moisture from the laminate polymer composite wound dressing can extend the maximum amount of fluid absorbed from the wound, because the amount of capacity lost to moisture can be minimized.

The moisture permeable material of the backsheet can include but is not limited to a polyurethane, such as Elastollan® 9109 (BASF, Florham Park, N.J.).

The laminate polymer composite wound dressing can further comprise an adhesive layer. Accordingly, in another embodiment, referring to the schematic cross-section view in FIG. 4, the laminate polymer composite wound dressing can comprise an absorbent layer 1 interposed between a contact layer 9 and a backsheet 11, and an adhesive layer 12 interposed between the absorbent layer 1 and the backsheet 11. The absorbent layer 1 comprises a fibrous web 2 and super absorbent polymer particles 3. The super absorbent polymer particles 3 are distributed throughout the absorbent layer 1 and are integral with the fibrous web 2. The fibrous web 2 is continuous, mostly oriented along the absorbent layer 1 and intersected with each other. The distance between horizontally adjacent intersection points 4 and 5 of the fibrous web 2 is larger than the distance between the vertically adjacent intersection points 6 and 7. The difference in the horizontal distance versus the vertical distance permits vertical expansion of the absorbent layer 1 upon exposure to wound exudates. The contact layer 9 comprises non-woven polymer fibers 10. Entanglement of the non-woven polymer fibers 10 of the contact layer 9 and the fibrous web 2 of the absorbent layer 1 binds a portion of the contact layer 9 to a portion of the absorbent layer 1 without the use of adhesives. For example, the non-woven polymer fibers 10 can be melt blown to the absorbent layer 1 without the use of adhesives. Optionally, a non-water soluble melt adhesive can be used to adhere the contact layer 9 to the absorbent layer 1. At least a portion of the backsheet 11 can be located on an opposite side of the absorbent layer 1 relative to the contact layer 9. The backsheet 11 comprises a moisture permeable material. The adhesive layer 12 comprises an adhesive and can be used to adhere at least a portion of the backsheet 11 to the absorbent layer 1.

The adhesive is not particularly limited so long as the adhesive can adhere one layer to another layer and allow for moisture to permeate through the adhesive layer. The adhesive of the adhesive layer can include a melt adhesive such as polyurethane or a pressure sensitive adhesive, wherein the adhesive can be structured to allow water vapor to permeate through the adhesive layer.

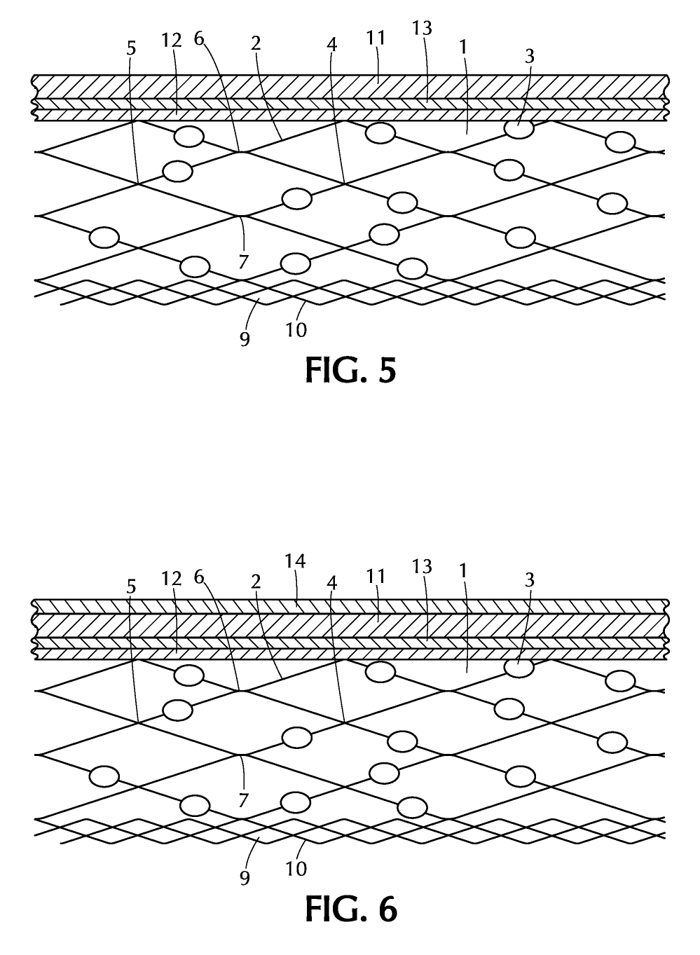

Exudation rates of the wound bed can vary for a variety of reasons (e.g., changes in the patient's blood pressure, infections, different healing rates, differences in wound bed contour, etc.). Because the amount and rate of exudates can change, the wound dressing needs to be able to accommodate a variety of exudate conditions without hampering the healing process. In other words, the wound dressing must be capable of adjusting its moisture vapor transmission control rate to accommodate very wet wounds, very dry wounds, and all moisture levels in between. Accordingly, the laminate polymer composition wound dressing can further comprise a moisture vapor transmission rate control layer. The moisture vapor transmission rate control layer appropriately absorbs exudates or hydrates at different rates at wound sub-zones that need the rates of liquid absorption and vapor releases into the ambient, but also that later on, automatically and adaptively self-adjusts to match changing wound conditions of the wound site and/or self-adjusts to match changing dimensions of the wound bed, while still promoting healing. Additionally, the moisture vapor transmission rate control layer helps to ensure that the wound dressing does not damage healthy skin surround the wound bed. Thus, in another embodiment, referring to FIG. 5, the laminate polymer composite wound dressing can comprise an absorbent layer 1 interposed between a contact layer 9 and a backsheet 11, an adhesive layer 12 interposed between the absorbent layer 1 and the backsheet 11, and an optional moisture vapor transmission rate control layer 13 interposed between the backsheet 11 and the adhesive layer 12. The absorbent layer 1 comprises a fibrous web 2 and super absorbent polymer particles 3. The super absorbent polymer particles 3 are distributed throughout the absorbent layer 1 and are integral with the fibrous web 2. The fibrous web 2 is continuous, mostly oriented along the absorbent layer 1 and intersected with each other. The distance between horizontally adjacent intersection points 4 and 5 of the fibrous web 2 is larger than the distance between the vertically adjacent intersection points 6 and 7. The difference in the horizontal distance versus the vertical distance permits vertical expansion of the absorbent layer 1 upon exposure to wound exudates. The contact layer 9 comprises non-woven polymer fibers 10. Entanglement of the non-woven polymer fibers 10 of the contact layer 9 and the fibrous web 2 of the absorbent layer 1 binds a portion of the contact layer 9 to a portion of the absorbent layer 1 without the use of adhesives. For example, the non-woven polymer fibers 10 can be melt blown to the absorbent layer 1 without the use of adhesives. Optionally, a non-water soluble melt adhesive can be used to adhere the contact layer 9 to the absorbent layer 1. At least a portion of the backsheet 11 can be located on an opposite side of the absorbent layer 1 relative to the contact layer 9. The backsheet 11 comprises a moisture permeable material. The adhesive layer 12 comprises an adhesive and can be used to adhere at least a portion of the backsheet 11 to the absorbent layer 1. The moisture vapor transmission rate control layer 13 is interposed between the backsheet 11 and the adhesive layer 12.

The moisture vapor transmission rate (MVTR) control layer is located between the backsheet and the adhesive layer. In order to remain on a highly exuding wound for a long period of time, wound dressings need to have a high MVTR. However, the wound dressing also needs to reduce vapor losses when used for dry wounds. Thus, the moisture vapor transmission rate control layer has a variable MVTR. When the MVTR control layer dries out, the moisture vapor transmission rate control layer permits no vapor transmission to protect dry wounds. When the MVTR control layer is wet with liquid exudate, however, moisture is able to permeate through the wound dressing. The moisture vapor transmission rate (MVTR) control layer can comprise polyvinyl alcohol (PVA), polyethylene oxide, polyvinyl pyrrolidone, and other known polymers and/or their blends or co-polymers with various degrees of cross-linkage breakability or hydrolyzation-ability being integrated into the characteristics of the picked polymer. In a specific embodiment, the moisture vapor transmission rate control layer comprises poly vinyl alcohol (PVA). Polyvinyl alcohol has a 60-80% degree of hydrolyzation, where the percent of hydrolyzation indicates what proportion of available polymer bonds are broken by prolonged exposure at temperature to a hydrolyzing solution (e.g. wound exudates).

The moisture vapor transmission rate control layer does not cover the entire area of the backsheet, there is an area left so that the backsheet can be adhered to the absorbent layer. The moisture vapor transmission rate control layer can be located in the middle of wound dressing. The moisture vapor transmission rate controls layer can cover about 90% of the backsheet.

The moisture vapor transmission control layer has an automatically variable MVTR; when the moisture vapor transmission rate control layer dries out (e.g. with a dry wound bed with limited wound exudate), the moisture vapor transmission rate control layer closes out, and there is no or limited vapor transmission. When the moisture vapor transmission rate control layer is wet (e.g. with highly exuding wounds), the moisture vapor transmission rate control layer allows moisture through. In other words, when the wound bed is relatively dry, the moisture vapor transmission rate control layer will exhibit a low MVTR, and when the wound bed is wet, the moisture vapor transmission rate control layer will exhibit a high MVTR and thereby automatically increase a moisture removal rate provided for the wound bed. Specifically, the moisture vapor transmission rate control layer may initially have low moisture vapor transmission rates (MVTR) such as <1000 g/m²/24 hours, preferably <500 g/m²/24 hours, when the material is dry (i.e. it has not yet been exposed to a hydrolyzing liquid, wound exudates), and it can have a substantially increased MVTR, for example >1000 g/m²/24 hours when the material has been exposed to a hydrolyzing solution (e.g. wound exudates) for a sufficiently long time duration. The wound dressing automatically and adaptively self-adjusts according to the degree of exudates absorbed from the wound bed and transmitted to the moisture vapor transmission rate control layer.

It is noted that moisture vapor transmission rate (MVTR) may be measured according to the German Institute for Standardization DIN EN 13726-2 standard.

In an embodiment, the laminate polymer composite wound dressing can further comprise at least one optional removable layers. The removable layers can be in the form of detachable strips, and can be vapor permeable but liquid impermeable. Laminations of thin polyurethane films may be used. Thus, in another embodiment, referring to FIG. 6, the laminate polymer composite wound dressing can comprise an absorbent layer 1 interposed between a contact layer 9 and a backsheet 11, an adhesive layer 12 interposed between the absorbent layer 1 and the backsheet 11, an optional moisture vapor transmission rate control layer 13 interposed between the backsheet 11 and the adhesive layer 12, and at least one removable layer 14 located on the opposite side of the backsheet 11 from the absorbent layer 1. The absorbent layer 1 comprises a fibrous web 2 and super absorbent polymer particles 3. The super absorbent polymer particles 3 are distributed throughout the absorbent layer 1 and are integral with the fibrous web 2. The fibrous web 2 is continuous, mostly oriented along the absorbent layer 1 and intersected with each other. The distance between horizontally adjacent intersection points 4 and 5 of the fibrous web 2 is larger than the distance between the vertically adjacent intersection points 6 and 7. The difference in the horizontal distance versus the vertical distance permits vertical expansion of the absorbent layer 1 upon exposure to wound exudates. The contact layer 9 comprises non-woven polymer fibers 10. Entanglement of the non-woven polymer fibers 10 of the contact layer 9 and the fibrous web 2 of the absorbent layer 1 binds a portion of the contact layer 9 to a portion of the absorbent layer 1 without the use of adhesives. For example, the non-woven polymer fibers 10 can be melt blown to the absorbent layer 1 without the use of adhesives. Optionally, a non-water soluble melt adhesive can be used to adhere the contact layer 9 to the absorbent layer 1. At least a portion of the backsheet 11 can be located on an opposite side of the absorbent layer 1 relative to the contact layer 9. The backsheet 11 comprises a moisture permeable material. The adhesive layer 12 comprises an adhesive and can be used to adhere at least a portion of the backsheet 11 to the absorbent layer 1. The optional moisture vapor transmission rate control layer 13 is interposed between the backsheet 11 and the adhesive layer 12. At least one of the optional removable layers 14 comprises a moisture permeable material. The moisture permeable material can have a moisture vapor transmission rate of from about 1.0 to about 3.0 kg/m²/day, is permeable to air, and substantially impermeable to a liquid and/or a bacteria.

A benefit to using a backsheet can be to prevent microorganisms such as bacteria from penetrating into the dressing. Another benefit to the backsheet can be that the moisture permeable material can regulate the moisture level of the dressing. For example, a moist environment can be important for wound healing. Therefore, for low exuding wounds, such as dry wounds, a high moisture vapor transmission rate can be undesirable. Thus, in one aspect, the laminate polymer composite wound dressings can comprise at least one optional removable layer. The removable layers can be in contact with the backsheet and can be made of the same or a different material from the backsheet. The choice of material for the removable layers is not limited so long as each layer has a moisture vapor transmission rate of from about 1.0 to about 3.0 kg/m²/day, can be permeable to air, and substantially impermeable to a liquid and/or a bacteria. The removable layer is vapor permeable but liquid impermeable, and can be a multi-layer stack of films and non-woven fabrics permanently or detachable bonded together. A benefit of having at least one removable layer is that the combined moisture vapor transmission rate of the backsheet, and the removable layer(s) becomes low enough that a low exuding wound can be kept moist. In an embodiment, depending on the level of wound exudate, the removable layer can remain part of the laminate polymer composite wound dressing or can be peeled off and removed by the user. A benefit to removing/peeling off at least one of the removable layer(s) can be that the combined moisture vapor transmission rate becomes high enough to prevent a wound from becoming too moist.

In an embodiment, the laminate polymer composite wound dressing of any of the above described embodiments is sealed in a sterile package. The wound dressing can be sterilized by e-beam radiation at 25-35 kgrey. This energy range provides a dosage of radiation high enough to sterilize the wound dressing, however, it does not cause further crosslinking of the super absorbing particles of the absorbent layer. Further crosslinking of the super absorbing particles would lead to undesired effects on absorbency of the absorbent layer.

The present invention describes a process for producing a laminate polymer composite wound dressing comprising melt blowing a portion of a contact layer onto a portion of an absorbent layer, thereby binding a portion of the contact layer to a portion of the absorbent layer. The contact layer comprises non-woven polymer fibers. The absorbent layer comprises a fibrous web and super absorbent polymer particles. The temperature during the melt blowing step can be higher than a glass transition point of the non-woven polymer fibers.

The processing parameters of the melt blowing step are not particularly limited so long as the melt blowing step can produce non-woven polymer fibers capable of binding to the surface and/or material of the absorbent layer. In an embodiment, at least some of the non-woven polymer fibers of a portion of the contact layer are entangled with at least some of the fibrous web of a portion of the absorbent layer. Melt blowing can include the following steps: an extrusion step, a blowing step, and a binding step. The extrusion step occurs when pressure and/or heat is applied to a feedstock to force a polymer material through at least one nozzle to form non-woven polymer fibers. The blowing step occurs when the non-woven polymer fibers join with a high velocity stream of hot air, wherein the temperature of the high velocity stream of hot air can be higher than the glass transition temperature of the non-woven polymer fibers. The binding step occurs when the nonwoven polymer fibers are blown onto, or otherwise make contact with, a substrate. The substrate for the melt blowing step includes the absorbent layer of the laminate polymer composite wound dressing. The binding step may take place through entanglement alone, or the binding step can include the use of heat, solvent, or binding agents to facilitate binding.

In an embodiment, the materials for producing the laminate polymer composite wound dressing include those described above for the contact, absorbent, backsheet, adhesive, and moisture vapor transmission rate control layers of the laminate polymer product.

In an embodiment, when melt blowing the contact layer, the non-woven polymer fibers of the contact layer have an elongation at break of 500% or more when measured at 73° F. and comprise a polyolefin, a ethylene-propylene copolymer, or a polyurethane of a polyether or a polyester. Further, the polyurethane of a polyether or a polyester can be characterized by a glass transition temperature from about −60° F. to about 0° F. The ethylene-propylene copolymer can have a glass transition temperature from about 110° F. to about 125° F. The choice of polymer for the non-woven polymer fibers is generally not limited so long as the polymer can be melt blown to bind to the absorbent layer. In an embodiment, the process produces non-woven polymer fibers can be applied at a density from about 1 to about 50 grams per square meter, including about 2 to about 15 grams per square meter.

In an embodiment, the process can further comprise adhering a backsheet onto an adhesive layer, which is the same as adhering the adhesive layer to the backsheet. The process can further include adhering an adhesive layer onto the absorbent layer or the process can, optionally, include adhering the adhesive layer to a moisture vapor transmission rate control layer. The order of these steps is not particularly limited so long as the layers are made to adhere to one another.

The step of adhering the adhesive layer to the backsheet and the step of adhering the adhesive layer to the absorbent layer or the moisture vapor transmission rate control layer can depend on the type of the adhesive present in the adhesive layer. For example, heat, pressure, and/or light may be applied to the layers or to the adhesive of the adhesive layer to facilitate adhesion between the layers.

In an embodiment, the process can, optionally, include the step of adhering a moisture vapor transmission rate control layer onto, including directly into contact with, the backsheet. The moisture vapor transmission rate control layer can be located between the backsheet and the adhesive layer.

In an embodiment, the process for producing a laminate polymer composite wound dressing further comprises treating at least one of the contact layer and the absorbent layer with at least one of sterile water, a polyol, an antimicrobial agent, a preservative, or a mixture thereof. In an embodiment, the process for producing a laminate polymer composite wound dressing can further comprise treating the absorbent layer with a solution of water, a preservative, such as bronopol (2-bromo-2-nitropropane-1,3-diol), and/or at least one polyol. Exemplary polyols are described elsewhere herein. A benefit to this treating step is to provide a moist wound dressing. A benefit of having a moist wound dressing is for application to dry wounds, where the wound dressing can support autolytic debridement. Autolytic debridement refers to the dissolving, or at least softening of, necrotic tissue to reduce the negative effects necrotic tissue has on the wound healing process. Treating one of the contact layer or the absorbent layer with at least one of sterile water, a polyol, an antimicrobial agent, a preservative, or a mixture thereof can have the benefit of supporting autolytic debridement by ensuring that the wound can be moist and that the laminate polymer composite wound dressing can be soft and flexible.

The process can further comprise adhering at least one removable layer directly to the backsheet, wherein at least one of the removable layers comprises a moisture permeable material, wherein the moisture permeable material has a moisture vapor transmission rate of from about 1.0 to about 3.0 kg/m²/day, can be permeable to air, and is substantially impermeable to a liquid and/or a bacteria.

A benefit to adhering a backsheet to the absorbent layer, or adhering a backsheet to an adhesive layer, or adhering a backsheet to a moisture vapor transmission rate control layer, can be the prevention of microorganisms, such as bacteria, from penetrating into the dressing. Another benefit to adhering the backsheet layer to the absorbent layer, or adhering a backsheet to an adhesive layer, or adhering a backsheet to a moisture vapor transmission rate control layer can be that the moisture permeable material can regulate the moisture level in the dressing. For example, a moist environment can be important for wound healing. Therefore, for low exuding wounds, a high MVTR can be undesirable. Thus, the laminate polymer composite wound dressing can comprise a moisture vapor transmission rate control layer, and, optionally, at least one removable layer. The moisture vapor transmission rate control layer can be adhered to the backsheet and the adhesive layer. The moisture vapor transmission rate control layer has a variable MVTR. The optional at least one removable layer can be adhered to directly to the backsheet. The at least one removable layer can be made of the same or a different material from the backsheet and from each other. The choice of the material for the at least one removable layer is not limited so long as at least one of the at least one removable layer(s) has a moisture vapor transmission rate of from about 1.0 to about 3.0 kg/m²/day, can be permeable to air, and is substantially impermeable to a liquid and/or a bacteria.

A benefit to adhering at least one of the at least one removable layer(s) to the backsheet can be that the combined moisture vapor transmission rate of the backsheet and at the at least one removable layer becomes low enough that the low exuding wound is kept moist. A benefit to removing/peeling off at least one of the removable layer(s) can be that the combined moisture vapor transmission rate, which includes the backsheet, becomes high enough to prevent a wound from becoming too moist. In an embodiment, depending on the level of wound exudate, at least one of the at least one removable layers can remain part of the laminate polymer composite or be peeled off or removed by the user.

In a further embodiment, the process can further comprise a sterilization step, wherein the wound dressing is provided in a sterilized form. In an embodiment, the wound dressing can be sterilized by e-beam radiation at 25-35 kgrey. This energy range provides a dosage of radiation high enough to sterilize the wound dressing, however, it does not cause further crosslinking of the super absorbing particles. Further crosslinking of the super absorbing particles would lead to undesired effects on absorbency of the absorbent layer.

To apply the wound dressing to a wound, the user (e.g. health care provider) opens the sterile pouch and orients the wound dressing so that the contact layer faces the wound. The wound dressing is then positioned so that the contact layer is against the wound and the backsheet or removable strips are exposure to air. The wound dressing can be positioned so that the center of the wound bed approximately coincides with the center of the wound dressing.

All cited patents and publications referred to in this application are herein incorporated by reference in their entirety for all purposes.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as disclosed. Without intending to limit the invention in any manner, embodiments will be more fully described by the following examples.

### EXAMPLES

### Example 1

A contact layer of melt-blown polyethylene polypropylene copolymer (Vistamaxx™ 2330 or 2330) is adhered directly onto an absorbent layer using a polyurethane adhesive. The absorbent layer includes a superabsorbent fleece (Luquafleece™ 402C, BASF). The amount of melt-blown polymer in the contact layer is about 5-20 grams per square meter. On the opposite side of the absorbent layer from the contact layer is located a moisture vapor transmission rate control layer comprising polyvinyl alcohol (PVA). The moisture vapor transmission rate control layer has a variable MTVR that automatically self-adjusts based on the moisture content of the wound exudate. An adhesive layer includes a pressure sensitive adhesive, such as RX 650 (Scapa), and is adhered to the moisture vapor transmission rate control layer and to the absorbent layer. A backsheet is located on the opposite side of the absorbent layer from the contact layer. The backsheet is a polyurethane film (Elastollan™ 9109) having a 10-30 micrometer thickness. The backsheet is corona treated in order to improve adhesion to the moisture vapor transmission rate control layer. The contact layer contains about 0.3% of PHMB as an antimicrobial additive. The addition of the antimicrobial additive to the contact layer is accomplished by adding the antimicrobial additive during the melt blown extrusion process while the absorbent layer is treated with a solution containing about 50-60% water, 30-40% of glycerine, 0.04% Bronopol.

### Example 2

A contact layer is be melt-blown polyurethane (Elastollan™ P 9291 or B 95A11n) adhered directly onto the absorbent layer, which includes a superabsorbent fleece (Luquafleece™ 402C, BASF). The amount of melt-blown polymer present in the contact layer is about 5-20 grams per square meter. On the opposite side of the absorbent layer from the contact layer is located a moisture vapor transmission rate control layer comprising polyvinyl alcohol (PVA). The moisture vapor transmission rate control layer has a variable MTVR that automatically self-adjusts depending on the moisture content of the wound exudate. The adhesive layer includes a pressure sensitive adhesive, such as RX 650 (Scapa) and is adhered to the moisture vapor transmission rate control layer and to the absorbent layer. A backsheet is located on the opposite side of the absorbent layer from the contact layer. The backsheet is a polyurethane film (Elastollan™ 9109) having 10-30 micrometer thickness. The backsheet is corona treated to improve adhesion to the moisture vapor transmission rate control layer. The contact and absorbent layers contain about 0.15% of triclosan (BASF) an antimicrobial additive. The addition of an antimicrobial additive to the contact layer is accomplished by adding the antimicrobial additive during the melt blown extrusion process while the absorbent layer is treated with a solution containing about 50-60% water, 30-40% of glycerine, 2-8% of Lutrol® F127 (BASF) and 2% of triclosan.

### Example 3

A wound care dressing made of synthetic polymers was produced. The wound care dressing consists of gelling polyacrylate (PA) fibers supported by polyester (PET) fiber mesh (the absorbent layer), enclosed between a polyurethane (PU) fiber contact layer and a polyurethane (PU) backsheet. When the wound care dressing is in contact with breached skin/wound and absorbs wound exudates, the polymer fibers of the absorbent layer form a gel. The gelling fibers are supported by a polyester (PET) fiber mesh inside the hydrophilic absorbing core, a polyurethane (PU) fiber wound contact layer on one side of the wound dressing, and a backsheet/moisture vapor transmission rate control layer of polyurethane (PU) and poly(vinyl alcohol)(PVA) films on another side of the wound dressing. Altogether, the components of the wound dressing provide structural integrity and allow for simple one-piece removal from the wound bed.

The backsheet of polyurethane film has no pores and is impermeable to fluids and microorganisms. The backsheet prevents fluid strike-through and serves as a microbial barrier.

To enhance softness of the wound care dressing, a small amount of a water/glycerin mixture (<5% of the absorbing capacity) is added to the polyacrylate (PA) and polyester (PET) fibers of the absorbent layer. For the purpose of preventing spoilage of this softening mixture during the manufacture of the wound dressing, Bronopol (2-bromo-2-nitropropane-1,3-diol), a commonly used preservative, in the amount of 0.04 wt % is added.

The wound dressing can be marketed as finished medical devices in a variety of sizes, e.g. 5*5 cm, 10*10 cm, 15*15 cm, and 15*25 cm (2*2, 4*4, 6*6, and 6*10 inches). The average thickness of the wound dressings is 3000±300μ, with a thickness distribution per layer (relative to the whole thickness) shown in Table 1.

TABLE 1

| | | Thickness | % of Total |
|---|---|---|---|
| Functional Layer | Material | (microns) | Thickness |
| Contact Layer | Polyurethane | 20 | 0.7 |
| Absorbent Layer | Polyacrylate | 2880 | 96 |
| | (PA)/Polyestyer | | |
| | (PET) fibers Glycerin/ | | |
| | water + preservative | | |
| Backsheet | Polyurethane | 20 | 0.7 |
| (with Moisture | (PU) film | | |
| vapor transmission | Polyvinyl alcohol | 80 | 2.6 |
| rate control layer) | (PVA) layer | | |
| | TOTAL THICKNESS: | 3000 | 100 |

The hydrophilic absorbing core fibers (i.e. absorbent layer) contain glycerin/water (<5% of absorbing capacity), with a preservative (Bronopol) in the amount of:

### Glycerin: 10 wt %;

### Water: 30 wt %;

### Preservative Bronopol: 0.04 wt %.

The wound dressing was manufactured by lamination of non-woven fiber layers and films. The technology is conventional roll-goods lamination utilized by medical device converting contract manufacturing companies. After lamination of all structural layers, the dressings were die-cut to the needed size using rotary die cut equipment. The softening glycerin/water/preservative solution was applied in a controlled manner at room temperature to each wound dressing before packaging into the heat-sealed foil pouches.

The hydrophilic absorbing core (i.e. absorbent layer) is produced by BASF, Inc. under the trade name Luquafleece®. Luquafleece® is made by combining polyacrylate (PA) and polyester (PET) polymer non-woven fibers.

Each wound dressing can be packaged in a peel-open foil pouch and terminally sterilized by e-beam radiation to achieve a 10⁻⁶ Sterility Assurance Level (SAL).

The wound dressing is placed on a wound with the wound contact layer facing the wound. Upon absorption of the wound exudates, the dry polyacrylate (PA) polymer fibers of the absorbent layer transform into a gel. This gelling property facilitates a high exudates absorption and retention capability.

One skilled in the art will recognize that various modifications and variations can be made to the present invention without departing from the spirit or scope of the invention. Thus, it is intended that the present invention cover modifications and variations of this invention provided they come within the scope of the appended claims and their equivalents.

What is claimed is:

1. A laminate polymer composite wound dressing for placement over a wound comprising:
a contact layer comprising non-woven polymer fibers;
an absorbent layer comprising a fibrous web and super absorbent polymer particles that form a gel upon contact with liquid wound exudate, the absorbent layer being expandable and more permeable to a fluid in a direction substantially perpendicular to the wound than in a direction substantially parallel/horizontal to wound, the absorbent layer being able to expand in a direction perpendicularly to the wound to enable the wound dressing to conform to a wound having a varying depth;
a backsheet, wherein at least a portion of the backsheet is located on an opposite side of the absorbent layer relative to the contact layer, and wherein the backsheet comprises a moisture permeable material, wherein the backsheet is permeable to air and substantially impermeable to a liquid and/or a bacteria; and
a moisture vapor transmission rate control layer, disposed between the absorbent layer and the backsheet, wherein when the moisture vapor transmission rate control layer is wet with liquid wound exudate, moisture permeates through the moisture vapor transmission rate control layer, and when the moisture vapor transmission rate control layer is dry, no vapor transmits through the moisture vapor transmission rate control layer.

2. The laminate polymer composite wound dressing of claim 1, wherein the fibrous web comprises a polyester, and the super absorbent polymer particles comprise polyacrylate.

3. The laminate polymer composite wound dressing of claim 1, further comprising at least one removable layer, wherein removal of at least a portion of the removable layer results in a moisture vapor transmission rate for the polymer composite that increases or decreases the moisture vapor transmission rate prior to removal of the removable layer.

4. The laminate polymer composite of claim 3, wherein the at least one removable layer has a moisture vapor transmission rate of from 1.0 to 3.0 kg/m²/day.

5. The laminate polymer composite of claim 3, wherein said at least portion of the removable layer comprises a strip.

6. The laminate polymer composite of claim 1, wherein the backsheet material has a moisture vapor transmission rate of from 1.0 to 3.0 kg/m²/day.

7. The laminate polymer composite wound dressing of claim 2, wherein the moisture vapor control layer is selected from polyvinyl alcohol, polyethylene oxide, polyvinyl pyrrolidone and blends or copolymers thereof.

8. The laminate polymer composite wound dressing of claim 1, wherein the absorbent layer is at least about two times more permeable to a fluid in a direction substantially perpendicular/vertical to the absorbent layer than in a direction substantially parallel/horizontal to the absorbent layer.

9. The laminate polymer composite wound dressing of claim 1, wherein the moisture vapor transmission rate control layer has a moisture vapor transmission rate exceeding 1000 g/m²/24 hours when the wound dressing is in contact with a highly exuding wound.

10. The laminate polymer composite wound dressing of claim 1, wherein the moisture vapor transmission rate control layer has a moisture vapor transmission rate that varies depending upon the type of a wound being treated.

11. The laminate polymer composite of claim 3, wherein removal of the removable layer results in reduced a moisture vapor transmission rate of the composite so that a low exuding wound can be kept moist.

12. The laminate polymer composite of claim 1, wherein the absorbent layer contains water and glycerin.

13. The laminate polymer composite of claim 1, wherein the absorbent layer comprises an antimicrobial.

14. The laminate polymer composite of claim 7, wherein the backsheet comprises polyurethane.
